SEQUENCE LISTING (11 sequences)

SEQ ID NO: 1 - DNA, 117 bp, Artificial Sequence
  Multiple Cloning Sites of the pET21a vector
aaggagatatacatatggctagcatgactggtggacagcaaatgggtcgcggatccgaattcgagctccgtcgacaagcttgcggccgcactcgagcaccaccaccaccaccactga

SEQ ID NO: 2 - DNA, 45 bp, Artificial Sequence
  SDM primers LUBT168
tagcatgactggtggacagtaaatgggtcgcggatccgaattcga

SEQ ID NO: 3 - DNA, 45 bp, Artificial Sequence
  SDM primers LUBT169
tcgaattcggatccgcgacccatttactgtccaccagtcatgcta

SEQ ID NO: 4 - DNA, 42 bp, Artificial Sequence
  LUBT 75 primer of GFP gene
tcccccatggtacccgggagtaaaggagaagaacttttcact

SEQ ID NO: 5 - DNA, 42 bp, Artificial Sequence
  LUBT 76 primer for GFP gene
ccgccggtcgacctgcagttatttgtagagctcatccatgcc

SEQ ID NO: 6 - DNA, 38 bp, Artificial Sequence
  LUBT 188 primer
gcttttcccgttatccggatcacatgaaacggcatgac

SEQ ID NO: 7 - DNA, 38 bp, Artificial Sequence
  LUBT 189 primer
gtcatgccgtttcatgtgatccggataacgggaaaagc

SEQ ID NO: 8 - DNA, 42 bp, Artificial Sequence
  LUBT 128 forward primer for GFP screening
ccgccggaattcgtacccgggagtaaaggagaagaacttttc

SEQ ID NO: 9 - DNA, 36 bp, Artificial Sequence
  LUBT 129 reverse primer for GFP screening
ccgccggaattcttatttgtagagctcatccatgcc

SEQ ID NO: 10 - DNA, 39 bp, Artificial Sequence
  LUBT 009 Forward primer for Staphylokinase gene
ccgccggaattccatatgtcaagttcattcgacaaagga

SEQ ID NO: 11 - DNA, 39 bp, Artificial Sequence
  LUBT 187 Reverse primer for Staphylokinase gene with amber STOP 5′
ccgccggaattcaagcttctatttcttttctataacaac

(19) United States
(12) Patent Application Publication
(10) Pub. No.: US 2011/0165583 A1
(43) Pub. Date: Jul. 7, 2011

(54) VECTOR FOR IDENTIFICATION, SELECTION AND EXPRESSION OF RECOMBINANTS

(75) Inventors: Anjali Apte Deshpande, Maharashtra (IN); Sampali Banerjee, Maharashtra (IN); Jitendra Kumar, Maharashtra (IN); Sriram Padmanabhan, Maharashtra (IN)

(73) Assignee: Lupin Limited, Maharashtra (IN)

(21) Appl. No.: 13/061,640

(22) PCT Filed: Sep. 2, 2009
(86) PCT No.: PCT/IN2009/000482

§ 371 Date: Mar. 1, 2011

(30) Foreign Application Priority Data
Sep. 2, 2008 (IN) 1506/KOL/2008

(51) Int. Cl. C12Q 1/68; C12N 15/74; C12N 15/63

(52) U.S. Cl. 435/6.15; 435/471; 435/320.1

(57) ABSTRACT

A modified vector comprising a reporter gene having a STOP codon upstream of the multiple cloning site of the vector which is characterized in that the recombinant clones show fluoresce or show color in presence of inducer. A method for identification and selection of recombinant clones comprising the modified vector wherein the recombinant clones florescence or show color in a suitable suppressor strain of the STOP codon associated with the gene of interest. A method of preparation of recombinant clone comprising gene of interest and modified vector comprising amplification of gene of interest using specific primers containing STOP codon different from STOP codon used with reporter gene; cloning the amplified gene of interest in the modified vector; transformation of cloned modified vector in the STOP codon suppressor host cell wherein the STOP codon suppressor host cell is specific for STOP codon used with the gene of interest wherein the recombinant clones either fluorescence or show color depending upon the reporter gene used.

### FIELD OF INVENTION

The present invention relates to a modified vector prepared by ligating a reporter gene having a STOP codon upstream of the multiple cloning site of the vector whereby the modified vector when introduced in the host cell does not fluoresce or show color. The invention also relates to method of producing the modified vector and method of identification and screening of recombinant clone. It also relates to kit comprising the said vector.

### BACKGROUND OF THE INVENTION

Molecular cloning is an important tool in our endeavor to understand the structure, function and regulation of individual genes and their products. Molecular cloning provides a means to exploit the rapid growth of bacterial cells for producing large amounts of identical DNA fragments, which alone have no capacity to reproduce by themselves. The power of molecular cloning is remarkable: a liter of bacterial cells engineered to amplify a single fragment of cloned human DNA can produce about ten times the amount of a specific DNA segment that could be purified from the total cellular content of the entire human body.

A recombinant DNA consists of two parts: a vector and the passenger sequence that is the gene of interest (GOI). Vectors contribute in replication functions due to presence of origin of replication sequences in the vector. The process of joining the vector and passenger DNAs is called ligation. T4 DNA ligase enzyme carries out ligation process by using ATP energy to make the phosphodiester bond between the vector and passenger sequence. If the vector and passenger DNA fragments are generated by the action of the same restriction endonuclease, they will join by base-pairing due to the compatibility of their respective ends. Such a construct is then transformed into prokaryotic cell, where unlimited copies of the construct and essentially the passenger sequence are made inside the cell.

Next step is to screen and identify recombinant clones from non-recombinants. There are numerous methods developed to screen recombinant clones and the methods include polymerase chain reaction (PCR), blue white screening, vectors carrying lethal gene which gets inactivated upon insertion of any foreign gene, use of reporter gene which upon cloning, either fluoresce or the color disappears and other methods in the art.

The reporter gene which are commonly used are chloramphenicol acetyl transferase gene (CAT), .beta.-galactosidase gene (.beta.-gal), luciferase gene (luc), alkaline phosphatase gene (AP), secreted alkaline phosphatase gene (SEAP), beta-glucuronidase gene (GUS). All fluorescent protein gene (Green fluorescent protein, Yellow fluorescent protein, Red fluorescent protein, Enhanced green fluorescent protein, Orange fluorescent protein, Cyan fluorescent protein,), human growth hormone gene (hGH) and beta-lactamase gene (.beta.-lac). GFPs from other sources like Renilla and from Ptilosarcus may also be used. Host cells, including bacterial, yeast and mammalian host cells, and plasmids for expression of the nucleic acids encoding each luciferase and GFP and combinations of luciferases and GFPs may also be used in these hosts which by substitution of codons optimized for expression in selected host cells or hosts, such as humans and other mammals, or can be mutagenized to alter the emission properties.

On the other hand, various cloning vectors permitting direct selection (positive selection) of recombinant strains have been described in the scientific literature.

The process of screening bacterial transformants that carry recombinant plasmids (having gene of interest) is made more rapid and simple by the use of vectors with visually detectable reporter genes. The blue-white screen is one such molecular technique that allows for the detection of successful ligations in vector based gene cloning (Gronenborn and Messing, 1978). The molecular mechanism underlying this technique is based on genetic engineering of the lac operon present in the laboratory strain of Escherichia coli that serves as a host combined with a subunit complementation achieved with the cloning vector.

For example, the vector pBLUEscript, which is commercially available encodes a subunit of LacZ protein with an internal multiple cloning site (MCS), while the chromosome of the host strain encodes the remaining O subunit to form a functional beta galactosidase enzyme which is involved in lactose metabolism. As such, selecting right type of vector and competent cells are critical considerations to blue white screening. The multiple cloning site (MCS) can be cleaved by different restriction enzymes so that the foreign DNA can be inserted within the lacZ gene, thus disrupting the production of functional beta galactosidase. When E. coli cells containing just the vector are grown in the presence of an artificial substrate X gal (5-bromo-4-chloro-3-indolyl R galactoside), a colourless modified galactose sugar that is metabolized by Beta galactosidase, the colonies turn blue, due to production of active enzyme that gives rise to blue end product. On the other hand, when the piece of foreign DNA is inserted into the MCS, the lacZ gene cannot make an active protein fragment and thus functional beta galactosidase, as a result, colonies of the bacteria that contain cloned foreign DNA appear whitish. Isopropyl b D-1-thiogalactopyranoside (IPTG), which functions as the inducer of the Lac operon, can be used in some strains to enhance the blue phenotype.

The blue white screen technique involves a screening procedure (discrimination) rather than a procedure for selecting the clones. Discrimination is based on identifying the recombinant within the population of clones on the basis of a color. The LacZ gene, in the vector used for generating recombinants, may be non-functional and cannot produce beta-galactosidase. As a result, these cells cannot convert X-gal to the blue substance so the white colonies seen on the plate may not be recombinants but just the background vector and thus give false results. Moreover, this complex procedure requires the use of the substrate X-gal which is very expensive, unstable and is cumbersome to use.

Although the lacZ and many other systems have been extensively used for Gram negative bacteria like E. coli, there are limited options available for screening recombinants transformed in Gram positive bacteria.

Chaffin and Rubens in 1998 have developed a Gram-positive cloning vector pJS3, that utilizes the interruption of an alkaline phosphatase gene, phoZ, to identify recombinant plasmids. A multiple cloning site (MCS) was inserted distal to the region coding for the putative signal peptide of phoZ. Alkaline phosphatase expressed from the derivative phoZ gene (phoZMCS) retained activity similar to that of the native protein and cells displayed a blue colonial phenotype on agar containing 5-bromo-4-chloro-3-indolyl phosphate (X-p). Introduction of foreign DNA into the MCS of phoZ produced a white colonial phenotype on agar containing X-p and allowed discrimination between transformants containing recombinant plasmids versus those maintaining self-annealed or uncut vector. This cloning vector has improved the efficiency of recombinant DNA experiments in Gram-positive bacteria.

Another method for screening and identification of recombinant clones is by using the green fluorescent protein (GFP) obtained from jellyfish Aqueorea victoris. It is a reporter molecule for monitoring gene expression, protein localization, protein-protein interaction. GFP has been expressed in bacteria, yeast, slime mold, plants, drosophila, zebrafish and in mammalian cells. Inouye et al (1997) have described a bacterial cloning vector with mutated Aequorea GFP protein as an indicator for screening recombinant plasmids. The pGREENscript A when expressed in E. coli produced colonies showing yellow color in day light and strong green fluorescence under long-UV. Inserted foreign genes are selected on the basis of loss of the fluorescence caused by inactivation of the GFP production.

It has been observed that false results are associated while using fluorescence technique by using GFP gene or any other reporter gene with fluorescence and color technique using lacZ gene (Blue white screen technique) for screening and identification of recombinant clones. Blue white screen technique gives white colonies to the recombinant clones and blue colonies to non-recombinants. But along with it some light blue colonies are also found. We have also observed that the white colonies may not contain the recombinant clones. Thus this technique does not provide accurate screening gives approximately 5-10% of false positives (Godisak R et al, Beyond pUC: Vectors for cloning unstable DNA). This technique is cumbersome as for enhancing blue color the plates should be kept at 40 C. Also this technique requires usage of expensive inducers like Xgal and IPTG. Similar false results have been found when GFP gene and its variants are used as in fluorescent technique for screening and identification of recombinant clones.

Thus the commonly used method for screening and identification of recombinant clones are associated with problems of false positive results. Thus there is need to overcome all the disadvantages associated with the existing techniques of screening and identification of recombinant clones. Further there is need to provide vectors which would act both as cloning vectors and expression vectors.

US20060099673 discloses novel recombinant gene expression method by stop codon suppression. It describes a novel recombinant gene expression method based on a novel recombinant gene expression vector comprising a gene encoding a selectable marker protein which is separated by a translational stop signal from an upstream arranged gene of interest, whereby both genes are translationally linked. Consequently, the expression of said selectable marker gene may be reduced compared to the expression rate of said gene of interest. It also discloses expression of said gene of interest by using suppressor element (SECIS) in the construct to suppress the STOP codon. Further, due to natural error rate of ribosomes the fusion protein (protein of Gene of interest and reporter gene) is synthesized and fusion protein synthesis purely depends on the natural error rate of the host.

However the present invention uses two STOP codons. The STOP suppression is very much directive/dictative. The first STOP codon during selection of clones where specific suppressor cell line is used for transformation produces fusion protein, which aids in selection process depending on type of reporter gene used. The second STOP codon is used mainly for authentic protein of interest in non-suppressor cell line.

Thus the present invention overcomes the disadvantages associated with the prior art by constructing a new vector, which will make the cells fluoresce upon cloning and will be devoid of false positive results. This way a single clone can be used for screening and expression studies directly.

### OBJECTIVE OF THE INVENTION

Accordingly one objective of the present invention is modified vector prepared by ligating a reporter gene having a STOP codon upstream of the multiple cloning site where the modified vector when introduced in the host cell does not fluoresce or show color. Another object of the present invention is a method for identification and screening of recombinant clone comprising the gene of interest wherein the method involves replacing the STOP codon in the modified vector with gene of interest having a STOP codon different from STOP codon used with reporter gene whereby the recombinant clones fluoresce or show color in a suitable suppression strain of the STOP codon associated with the gene of interest and on expression in non suppressor strain do not fluorescence or show color and authentic protein of interest is obtained.

Another object of the present invention is a modified vector comprising reporter gene having a STOP codon upstream of the multiple cloning site of the vector.

Another object of the present invention is a method of preparing a modified vector comprising reporter gene having a STOP codon upstream of the multiple cloning site of the vector comprising:
(a) Introduction of STOP codon upstream of multiple cloning site of the vector
(b) Amplification of reporter gene using primers
(c) Cloning of reporter gene in the vector, wherein the modified vector when introduced in the non suppressor E. coli host does not fluorescence or show color when expressed upon induction.

Another object of the present invention is a method of preparation of recombinant clones comprising gene of interest and modified vector wherein the method comprises:
(a) Amplification of gene of interest using specific primers containing STOP codon other than the STOP codon used with reporter gene
(b) Cloning the amplified gene of interest in the modified vector
(c) Transformation of cloned modified vector in the STOP codon suppressor host cell specific for STOP used in gene of interest I wherein the recombinant clones either fluoresce or show color depending upon the reporter gene used.

Another object of the present invention is a kit for identification and expression of recombinant clones comprising modified vector wherein the modified vector comprise of reporter gene carrying STOP codon.

Another object of the present invention is a kit for indicating the solubility of foreign protein expressed using recombinant clone wherein foreign protein is expressed using a recombinant clone identified and screened using modified vector.

### SUMMARY OF THE INVENTION

Accordingly one aspect of the present invention relates to a modified vector prepared by ligating a reporter gene having a STOP codon upstream of the multiple cloning site of the vector whereby the modified vector when introduced in the host cell does not fluoresce or show color.

According to another embodiment of the present invention there is provided a method for identification and screening of recombinant clone comprising the gene of interest wherein the method involves the replacing the STOP codon in the modified vector with gene of interest having a STOP codon different from STOP codon used with reporter gene whereby the recombinant clones fluoresce or show color in a suitable suppression strain of the STOP codon associated with the gene of interest and on expression in non suppressor strain do not florescence or show color and authentic protein of interest is obtained.

According to another embodiment of the present invention there is provided a modified vector comprising reporter gene having a STOP codon upstream of the multiple cloning site of a vector.

According to another embodiment of the present invention there is provided a method of preparation of a modified vector comprising a reporter gene having a STOP codon upstream of the multiple cloning site of the vector comprising:
(a) Introduction of STOP codon upstream of multiple cloning site of the vector
(b) Amplification of reporter gene using primers
(c) Cloning of reporter gene in the vector, wherein the modified vector when introduced in the non suppressor E. coli host does not fluorescence or show color when expressed upon induction.

According to another embodiment of the present invention there is provided a method of preparation of recombinant clone comprising gene of interest and modified vector wherein the method comprises:
(a) Amplification of gene of interest using specific primers containing STOP codon different from STOP codon used with reporter gene
(b) Cloning the amplified gene of interest in the modified vector
(c) Transformation of cloned modified vector in the STOP codon suppressor host cell specific for STOP used in gene on interest wherein the recombinant clones either fluorescence or show color depending upon the reporter gene used.

According to another embodiment of the present invention there is provided a kit for identification and expression of recombinant clones comprising modified vector wherein the modified vector comprise of reporter gene carrying STOP codon.

According to another embodiment of the present invention there is provided a kit for indicating the solubility of foreign protein expressed using recombinant clone wherein foreign protein is expressed using a recombinant clone identified and screened using modified vector.

### BRIEF DESCRIPTION OF THE ACCOMPANYING DRAWINGS

FIG. 1: Plasmid map of the pET21a vector with TAA introduction upstream of MCS

FIG. 2: Restriction analysis of clones of pBAD24-GFP

FIG. 3: Plasmid map of pBAD24-GFP clone (pLUBT133)

FIG. 4: Release of GFP insert from pLUBT133

FIG. 5: Colony PCR screening for confirmation of pET21a GFP.

FIG. 6: Plasmid map of pET21a-GFP clone (pLUBT166)

FIG. 7: Colony PCR screening for confirmation of modified pET21a-GFP

FIG. 8: Plasmid map of pET21a modified-GFP clone ((pLUBT179)

FIG. 9: GFP expression in pET21a-GFP and pET21 modified-GFP clones

FIG. 10: Confirmation of NdeI modification in clones 5,6,7,8,20 by double digestion with enzymes NdeI/HindIII

FIG. 11: Plasmid map of pLUBT 189 FIG. 12: pLUBT189 digested with XbaI/HindIII

FIG. 13: Confirmation of GFP fragment insertion in pBAD24 by GFP PCR.

FIG. 14: Plasmid map of pLUB191

FIG. 15: SAK PCR with amber stop

FIG. 16: Glowing colonies are #1, 4, 8, 9, 10 etc while the non-glowing colonies are #5, 6, 18, 42 and 48.

FIG. 17: PCR for SAK gene and GFP gene

FIG. 18: SDS-PAGF of SAK-GFP fusion protein in non amber suppressor strain.

FIG. 19: Schematic representation for construction of pLUBT191 & 196

FIG. 20: Relative fluorescence intensities of fusion proteins SAK-GFP & GCSF-GFP

### DETAILED DESCRIPTION OF THE INVENTION

All the vectors described in the literature and the vectors available commercially have either the color disappearing by insertional inactivation or the fluorescence disappearing upon cloning. These methods can generate false positives results, as disappearance of color or fluorescence may not be completely achieved that is there could be a possibility of background color or fluorescence.

Thus the present invention involves construction of a modified vector for screening and identification of recombinant clones, where in the recombinant cells fluoresce or show color and non recombinants does not fluoresce or show color. This would avoid the false positive results associated with prior art techniques. This vector could further be used for expression studies.

For the present invention, the vector is selected from plasmid, phage, cosmid and the like with no particular limitation.

Vectors suitable to be used for the present invention are numerous and a list of the vectors can be found in the art. The vectors commercially available from Stratagene, Promega, CLONTECH, Invitrogen GIBCO Life Sciences and other companies making expression vectors. All the vectors with bacterial promoters may be used.

Vectors particularly suitable are plasmid vectors, which include prokaryotic, eukaryotic and viral sequences. A list of these vectors can be found in Gene Transfer and Gene Expression: A Laboratory Manual, Ed. Kriegler, M., Stockton Press, New York (1990) and Molecular Cloning, A Laboratory Manual, CSH Laboratory Press, Cold Spring Harbor, N.Y. and Current Protocols in Molecular Biology, Vol. 1, Supplement 29, section 9.66, Ed. Asubel, F. M. et al., John Wiley & Sons (2001).

The modified vector relates to a vector, which is modified to include a reporter gene with a STOP codon.

A codon is a group of three bases—A, T, C, or G—and codes for a single amino acid, the building blocks of proteins.

A STOP codon stops translating the code when ribosome reaches the end of the protein. STOP codons come in three different forms—TGA, TAG, and TAA. A STOP codon signals the cell's machinery that it has reached the end of the protein and should STOP translating the code. More preferably for the present invention the STOP codon is TAA only with reporter gene.

Reporter gene means a gene that is not endogenously expressed in the used cell type that is typically used to evaluate another gene, especially its regulatory region. Expression of reporter gene changes the phenotypic characteristic of the cell that carries the gene. Representative reporter genes are, .beta.-galactosidase gene (.beta.-gal), luciferase gene (luc), alkaline phosphatase gene (AP), secreted alkaline phosphatase gene (SEAP), .beta.-glucuronidase gene (GUS), All fluorescent protein gene (Green fluorescent protein, Yellow fluorescent protein, Red fluorescent protein, Enhanced green fluorescent protein, Orange fluorescent protein, Cyan fluorescent protein), substituted p-nitrophenyl phosphate and their variants.

Another embodiment of the present invention relates to a method for identification and screening of recombinant clone wherein the method involves ligating a reporter gene having a STOP codon upstream of the multiple cloning site of a vector to prepare a modified vector. The modified vector when introduced in the host cell do not fluorescence or show color due to the presence of STOP codon.

The reporter gene and the vector used can be any of those disclosed above and mentioned in the prior art.

The present invention involves a modified vector comprising reporter gene having a STOP codon upstream of the multiple cloning site of the vector.

Another embodiment of the present invention is to provide a method of preparation of modified vector comprising reporter gene having a STOP codon upstream of the multiple cloning site of a vector comprising:
(a) Introduction of STOP codon upstream of multiple cloning site of the vector
(b) Amplification of reporter gene using primers
(c) Cloning of reporter gene in the vector, wherein the modified vector when introduced in the non suppressor E. coli host does not fluorescence or show color when expressed upon induction.

In another embodiment of the present invention there is provided a method of preparation of recombinant clone comprising gene of interest and modified vector wherein the method comprises:
(a) Amplification of gene of interest using specific primers containing STOP codon different from the one used in modified vector
(b) Cloning the amplified gene of interest in the modified vector
(c) Transformation of cloned modified vector in the suppressor host cell specific to the STOP codon used with the gene of interest wherein the recombinant clones either fluorescence or show color depending upon the reporter gene used.

After identification of the recombinant clones, these recombinant clones were expressed using a non suppressor host cell. The recombinant clones does not fluorescence and show color and protein of interest is expressed.

The present invention involves introduction of a STOP codon upstream of multiple cloning site of the vector using site directed mutagenesis (SDM) primers wherein one of the codon was replaced with STOP codon. Any of the previously mentioned STOP codon can be used. STOP codon incorporation was confirmed using DNA sequence analysis. The most preferable STOP codon is TAA codon. The site directed mutagenesis could be performed by any of the methods known in the art.

The next step involves cloning the reporter gene in the vector to get the modified vector. First the reporter gene was amplified by using PCR technique and cloned into vector carrying STOP codon. The most preferred reporter gene for the present invention is GFP gene or beta.-galactosidase gene. The cloned modified vector i.e. the transformants were transformed in the host cell and the clones were examined for the presence of GFP insert by digestion and PCR. Also this reporter gene was inserted in the non-modified vector. The results indicate that the STOP codon interfered with GFP translation in modified vector whereas GFP translation occurred in non-modified vector. Thus the recombinant clones from the modified vector did not show florescence and in case of non-modified vector showed fluorescence under UV light radiation.

For cloning any foreign gene in bacterial expression system NdeI is the preferred restriction site as it provides start codon and avoids addition of extra amino acids at N terminus. In the constructed modified vector, there are two NdeI sites, one is present in MCS and required for cloning foreign gene and the other in GFP gene, which will interfere with the cloning strategy of foreign gene. Thus the NdeI site in the GFP gene was altered without altering the amino acid by Site Directed Mutagenesis. This vector along with modified NdeI site of GFP was used for cloning the gene of interest. It was confirmed by independent experiment that modification of NdeI site did not affect the glow of GFP.

The present invention involves a method for identification and screening of recombinant clones comprising the gene of interest wherein the method involves replacing the multiple cloning site of the vector and the STOP codon in the modified vector with gene of interest having a STOP codon different from the one used with reporter gene. The above vector comprising the gene of interest when introduced in the suppressor strain specific to the STOP codon used with the gene of interest fluoresce or shows color but when the identified recombinant clones are introduced in the suppressor cells for expression does not fluorescence or show color and authentic protein of interest is obtained.

The present invention involves the use of gene of interest known to the person skilled in the art at the time of invention.

It involves the cloning of foreign gene into the above-modified vector. Any gene of interest can be used. The present invention offers a cost effective process for screening and identification of recombinant clones comprising gene of interest. To exemplify the present invention Staphylokinase gene (SAK) was cloned.

Cloning of Staphylokinase gene carrying STOP codon different from STOP codon used with reporter gene at NdeI/EcoRI site of the modified vector to produce a GFP fusion protein. The most preferable STOP codon is TAG amber codon.

If the SAK gene got inserted in right frame the recombinants upon transfer to amber suppressor strains (Since TAG amber codon is used) would glow and could be selected.

When introduced in non-Amber suppressor strains, they would make only recombinant Staphylokinase and would not fluorescence or show color. The use of suppressor strains would depend upon the type of STOP codon used with the gene of interest.

The choice of bacterial cell line depends on the STOP codon, the various types of E. coli cells which may be used are amber suppressor, ochre suppressor and opal suppressor E. coli.

According to another embodiment of the present invention there is provided a kit for identification and expression of recombinant clones comprising modified vector wherein the modified vector comprise of reporter gene carrying STOP codon.

The modified vector according to the present invention is advantageously combined in a kit of parts (preferably, in a cloning and expression kit) with reporter gene and carrying a STOP codon.

A STOP codon stops translation of the code when ribosomes reach the end of the protein. STOP codons come in three different forms—TGA, TAG, and TAA. A STOP codon signals the cell's machinery that it has reached the end of the protein and should stop translating the code. Any of the STOP codon can be used. More preferably for the present invention the STOP codon used with the reporter gene to construct a modified vector is TAA and the STOP codon used with the gene of interest is the TAG.

The reporter genes may be, .beta.-galactosidase gene (.beta.-gal), luciferase gene (luc), alkaline phosphatase gene (AP), secreted alkaline phosphatase gene (SEAP), .beta.-glucuronidase gene (GUS), All fluorescent protein gene (Green fluorescent protein, Yellow fluorescent protein, Red fluorescent protein, Enhanced green fluorescent protein, Orange fluorescent protein, Cyan fluorescent protein), substituted p-nitrophenyl phosphate and their variants. Preferably green fluorescent protein gene (GFP) is used.

The kit of the present invention further comprise of gene of interest carrying a STOP codon different from STOP codon used with reporter gene. Any of the gene of interest mentioned in the prior art can be used.

This kit can be used for:
(1) False positives could be completely avoided
(2) Kit would provide a cost effective way of screening, identification and expression of the recombinant clones in two different bacterial cell lines
(3) This kit utilizes the property of colour or fluorescence to be obtained after cloning.
(4) This kit could be applicable to cloning of any size genes since reporter gene esp GFP is known to fluoresce when cloned as fusion protein with any size gene at the N terminus.
(5) This kit would save a great deal of time since only fluorescent clones (which are indicative of only recombinants) need to be processed for DNA preparation and expression studies thereon.

According to another embodiment of the present invention there is provided a kit for indicating the solubility of foreign protein expressed using recombinant clone wherein foreign protein is expressed using a recombinant clone identified and screened using modified vector.

It has been found that the intensity of GFP fluorescence is dependent on the solubility of GFP. It is brightest when expressed in soluble form and decreases with decrease in solubility. (Davis and Vierstra, 1998). Hence, the solubility of the protein of interest would have an effect on the solubility of fusion protein and thereby affect the GFP fluorescence intensity. Thus from the relative fluorescence intensity of the fusion protein one can qualitatively assess the solubility propensity of the protein of interest.

This is applicable to all other reporter gene.

Earlier reports have suggested that GFP expression is affected by OmpT proteases as there are two ompT protease sites in the GFP gene (Shi and Su, 2001). OmpT expression is reported to be low at 28 deg and hence GFP expression could be more pronounced at this temperature (Stathopoulous et al, 1999, Ogawa et al, 1995). There are also reported inhibitors of OmpT like zinc chloride and copper chloride at 0.1 to 0.5 mM final concentration (Baneyx and Georgieu, 1990).

LE 392 is an amber suppressor strain and is known to express lon protease and OmpT protease. To minimize the expression of these proteases which otherwise might interfere with GFP stability; we decided to use LE392 to express GFP with the following conditions after transformation.

After introduction of the foreign genes like SAK, the ligation mix was introduced into competent LE392 cells and then the plates were incubated at 30 deg C. instead of regular 37 deg C.

The preferred embodiments of the present invention are described with reference to the following non-limiting example:

GFP gene was expressed from a known T7 expression vector. STOP codon was introduced before the GFP gene in this vector, which resulted in non-glowing transformants but gave positive PCR for GFP. Then NdeI site in GFP gene was modified by site directed mutagenesis (SDM) where as NdeI site in the vector was available for cloning the foreign gene. This construct was used to clone foreign gene that carried Amber STOP codon at 3′ end and was cloned at NdeI site at 5′ of GFP gene. The GFP fragment along with MCS and necessary changes was subcloned in pBAD24 vector. This construct has inducible promoter which can be induced by relatively cheaper inducer for protein expression. Amber suppressor cell line like DH5 alpha, JM109 and LE392 were transformed with this construct.

Recombinants were screened by checking for the presence of glow under UV light and were then inoculated for DNA preparation. These DNAs were introduced into nonamber suppressor strains like BL21 series and then induced with the inducer to get the native protein of right size due to recognition of amber codon as a STOP codon in the current cell line. This way a single clone can be used for screening and expression studies directly.

### Example 1

### Introduction of STOP Codon into the MCS of pET21a Vector: (pLUBT176)

The STOP codon is of three types TAA, TAG and TGA. For this particular example TAA as a STOP codon is used. But the present invention can also be carried out using any other STOP codon and any other vector known in the prior art.

The MCS of the pET21a vector from Novagen, USA is as follows:

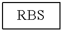

SEQUENCE ID 1
. . . AAGGAGATATA CAT ATG GCT AGC ATG ACT GGT GGA
CAG CAA ATG GGT CGC GGA TCC GAA TTC GAG CTC CGT
CGA CAA GCT TGC GGC CGC ACT CGA GCA CCA CCA CCA
CCA CCA CTG A . . .

The STOP codon was introduced into pET21a vector at the base (indicated as underlined) using the SDM primers LUBT168 and 169 by modifying the CAA to TAA. The sequence is as follows

LUBT168:
SEQUENCE ID 2
5′ T AGC ATG ACT GGT GGA CAG TAA ATG GGT CGC GGA
TCC GAA TTC GA 3′
LUBT169:
SEQUENCE ID 3
5′ TC GAA TTC GGA TCC GCG ACC CAT TTA CTG TCC ACC
AGT CAT GCT A 3′

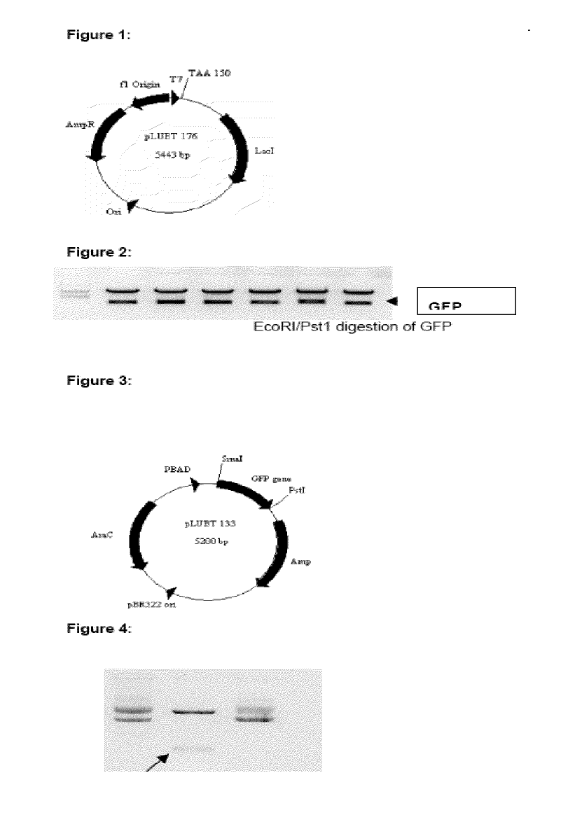

Site Directed Mutagenesis (SDM) was performed by using the Stratagene kit by using known methods. The SDM conditions were as follows: Initial denaturation at 95° C. for 30 sec followed by 15 cycles of 95° C. for 30 sec, 55° C. for 1 min and 68° C. for 6 min as per manufacturers instructions. After the SDM, the PCR reaction was subjected to Dpn1 digestion for 1 hr at 37 deg C. The reaction mixture was inactivated at 65 deg C. for 20 minutes, cooled and then introduced into competent DH5 alpha cells. TAA incorporation was confirmed by DNA sequence analysis of selected transformants. FIG. 1 shows the plasmid map of TAA introduction into the MCS of pET21a vector

### Example 2

### Cloning of Reporter Gene into pBAD24 Vector: (pLUBT133)

Reporter gene such as, beta.-galactosidase gene (.beta.-gal), luciferase gene (luc), alkaline phosphatase gene (AP), secreted alkaline phosphatase gene (SEAP), .beta.-glucuronidase gene (GUS), All fluorescent protein gene (Green fluorescent protein, Yellow fluorescent protein, Red fluorescent protein, Enhanced green fluorescent protein, Orange fluorescent protein, Cyan fluorescent protein) can be used. Any other reporter gene known in the prior can also be used. But for the present example, GFP gene is used as the reporter gene.

The GFP gene was PCR amplified using primers LUBT 75/LUBT 76 the sequences of which are given below:

LUBT 75:
SEQUENCE ID 4
5′ TCC CCC ATG GTA CCC GGG AGT AAA GGA GAA GAA
CTT TTC ACT 3′
LUBT 76:
SEQUENCE ID 5
5′ CCG CCG GTC GAC CTG CAG TTA TTT GTA GAG CTC
ATC CAT GCC 3′

The PCR amplification was performed with Taq DNA polymerase from Bangalore Genei Pvt. Ltd, Bangalore, India with following amplification conditions: After an initial denaturation time of 5 min @ 950 C, 30 cycles consisting of 950 C for 30 sec, 500 C for 30 sec and 720 C for 30 sec were continued. The amplified GFP gene was purified, digested with SmaI/PstI and cloned into pBAD24 vector (National Institute of Genetics, Japan) at similar sites. FIG. 2 shows restriction analysis of clones of pBAD24-GFP.

The transformants were directly inoculated for plasmid DNA preparation and the DNA from the clones were examined for the presence of GFP insert by digestion with suitable restriction enzymes.

### Example 3

### Subcloning of GFP as EcoRI/HindIII Fragment in pET21a and pET21a Modified Vector: (pLUBT166 and pLUBT179 Respectively)

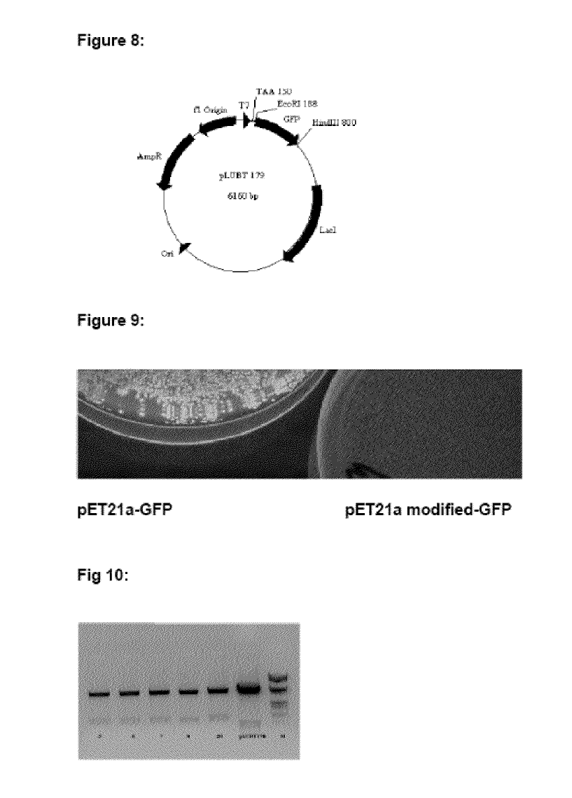

GFP was excised from pLUBT133 (FIG. 3) as EcoRI/HindIII (FIG. 4 shows release of GFP insert from pLUBT133) and cloned into pET21a (FIG. 6) and modified pET21a (FIG. 8) (with STOP codon TAA in MCS) into similar sites.

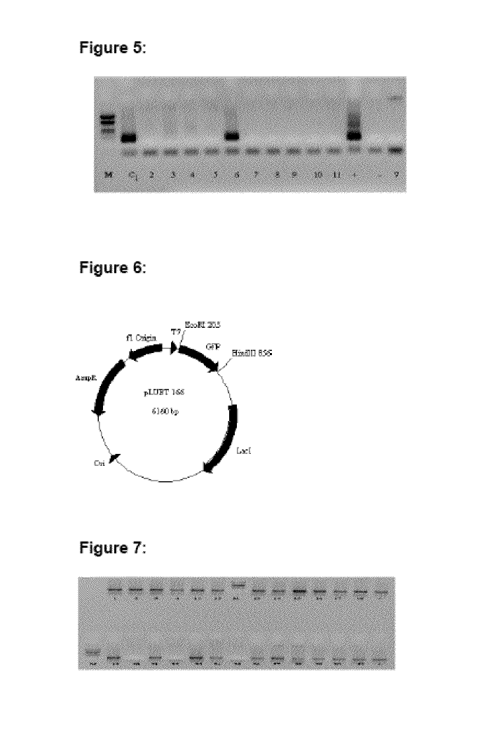

The clones of pET21a-GFP (FIG. 5) and pET21a modified-GFP (FIG. 7) were screened for GFP PCR. and DNAs of clones showing positive PCRs were introduced into T7 RNA polymerase expressing cells BL21A1 (Invitrogen, USA). The cells were then plated on LB/amp arabinose plates since arabinose acts as inducer for the T7 RNA polymerase in BL21 A1 cells.

It was found that pET21a-GFP shows florescence but pET21a-modified-GFP does not show florescence due to the presence of STOP codon (FIG. 9). The results indicate that the STOP codon TAA interfered with the GFP translation as expected and hence the colonies transformed with this DNA did not show any fluorescence while the same fragment in usual pET21a showed the glow under UV light.

### Example 4

### NdeI Site Modification of the GFP Gene by SDM

Site Directed Mutagenesis was done to modify the NdeI site which is present in the GFP gene and interfere with the cloning strategy of foreign gene. Thus the NdeI site in GFP gene of pLUBT179 clone was mutated by SDM using primers LUBT 188/189.

LUBT 188:
SEQUENCE ID 6
5′ GC TTT TCC CGT TAT CCG GAT CAC ATG AAA CGG
CAT GAC3′
LUBT 189:
SEQUENCE ID 7
5′ GTC ATG CCG TTT CAT GTG ATC CGG ATA ACG GGA
AAA GC 3′

SDM cycle
95° C.-30 sec
95° C.-30 sec
55° C.-1 min 15 cycles
68° C.-6 min

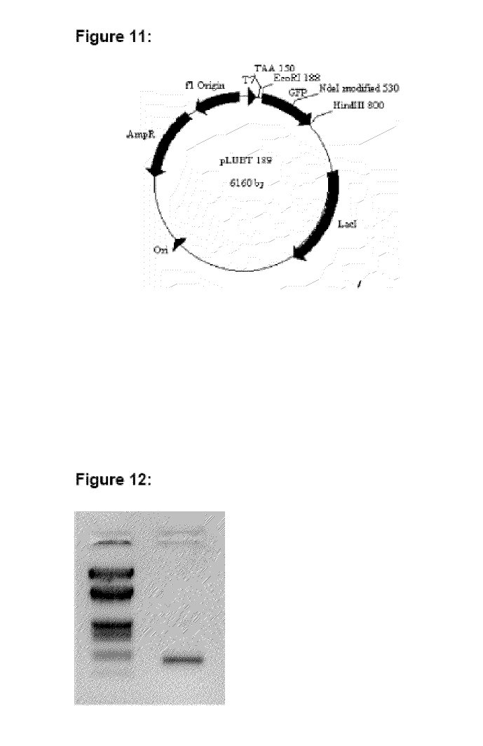

After SDM the ligation reaction mixture was introduced into DH5α competent cells to get modified pLUBT179 and transformants were confirmed by linearization with NdeI. The clones were further confirmed by double digestion with NdeI/HindIII (FIG. 10) and the new construct was labeled as pLUBT189 (FIG. 11).

### Example 4

### Introduction of NdeI Modified GFP into pBAD24 Vector

To prepare as prokaryotic expression vector under a commonly used promoter based system, the NdeI modified GFP under T 7 promoter was placed in vector pBAD24.

The pLUBT189 was digested with XbaI/HindIII to release the fragment (FIG. 12) having GFP gene (770 bp). It was then purified and ligated with NheI/HindIII digested pBAD24 vector and the ligation mix was used to transform DH5a competent cells.

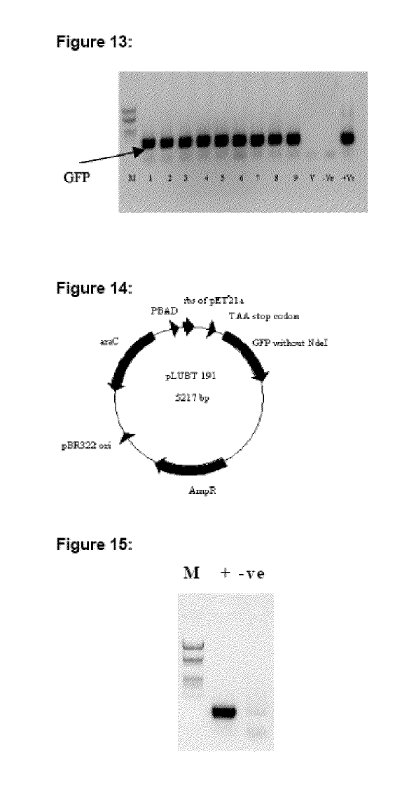

Transformants were screened by colony PCR using GFP specific primers LUBT128 and 129 as forward and reverse primer respectively. Several colonies were positive for GFP PCR (FIG. 13) but never showed any fluorescence in the presence of the inducer LUBT 128: forward primer for GFP screening—SEQUENCE ID 8

5′ CCG CCG GAA TTC GTA CCC GGG AGT AAA GGA GAA
GAA CTT TTC 3′

LUBT 129: reverse primer for GFP screening—SEQUENCE ID 9

5′ CCG CCG GAA TTC TTA TTT GTA GAG CTC ATC CAT
GCC 3′

The construct was designated as pLUBT191 (FIG. 14), has GFP under arabinose promoter but with TAA STOP at the N terminus giving a clone of GFP, which does not glow even in the presence of inducer.

### Example 5

### Cloning of Foreign Genes into the Above Vector

Any foreign gene can be used for cloning in the modified vector. For the present example GFP-STOP vector (LUBT 191) was used. The foreign gene used in the present example is Staphylokinase gene. However any other reporter gene containing vector can also be used for cloning foreign gene.

Cloning of Staphylokinase gene (SAK gene) carrying Amber STOP codon at 3′ end at NdeI/EcoRI site of the modified vector was carried out. If the SAK gene got inserted in right frame, the recombinant clones upon transfer to amber suppressor strains glow under UV and were screened and selected. When recombinant clone was introduced in nonamber suppressor strains, the clones do not glow and expresses only recombinant Staphylokinase. The step for cloning foreign gene is as follows.

Staphylokinase gene was amplified from synthetic genes using specific primers containing amber STOP codons and cloned into pLUBT191 as NdeI/EcoRI fragments.

### Primers for Amplification:

The primers for foreign genes must have the amber suppressor codon

LUBT 009: Forward primer for Staphylokinase
gene-
SEQUENCE ID 10
5′ CCG CCG GAA TTC CAT ATG TCA AGT TCA TTC GAC
AAA GGA 3′
LUBT 187: Reverse primer for Staphylokinase
gene with amber STOP 5′--
SEQUENCE ID 11
CCG CCG GAA TTC AAG CTT CTA TTT CTT TTC TAT AAC
AAC 3′

### PCR Conditions:

The Staphylokinase gene was PCR amplified using Taq DNA polymerase from Bangalore Genei Pvt. Ltd (Bangalore, India) with the following amplification conditions. Initial denaturation of 4 minutes at 94 deg c followed by 30 cycles of 94° C. for 30 sec, 57° C. for 30 sec and 72° C. for 30 sec. After a final extension of 7 min at 72° C., the PCR amplified product was checked on 1% agarose gel (FIG. 15), purified and then digested with NdeI/EcoRI and ligated to the GFP STOP vector pLUBT191 at similar sites.

The ligation mix of pLUBT191 and NdeI/EcoRI digested SAK fragments were transformed into competent LE392 cells which is an established amber suppressor strain with the following genotype glnV44 SupF58 (lacY1 or AlacZY) galK2 galT22 metB1 trpR55 hsdR514(rK−mK+).

The transformants were replica plated on LB agar plates containing 100 μg/ml ampicillin and 13 mM L(+) Arabinose.

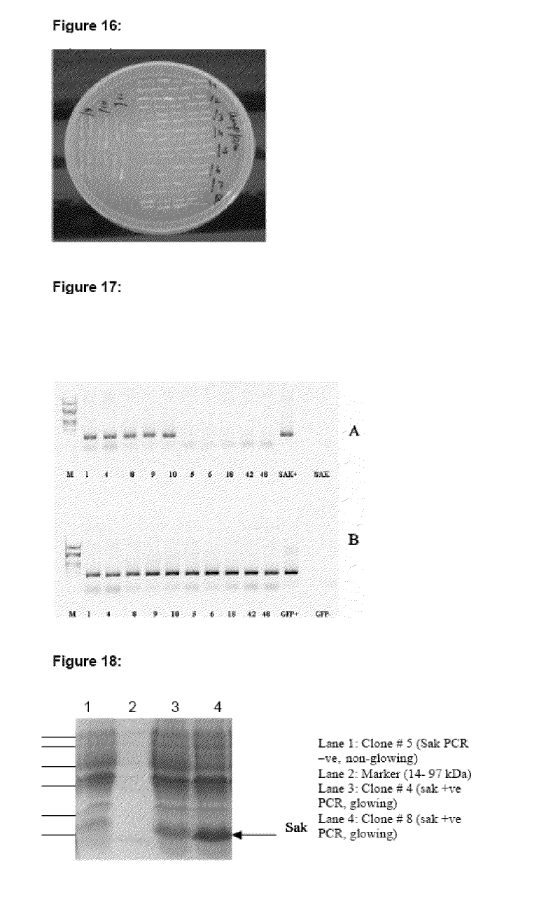

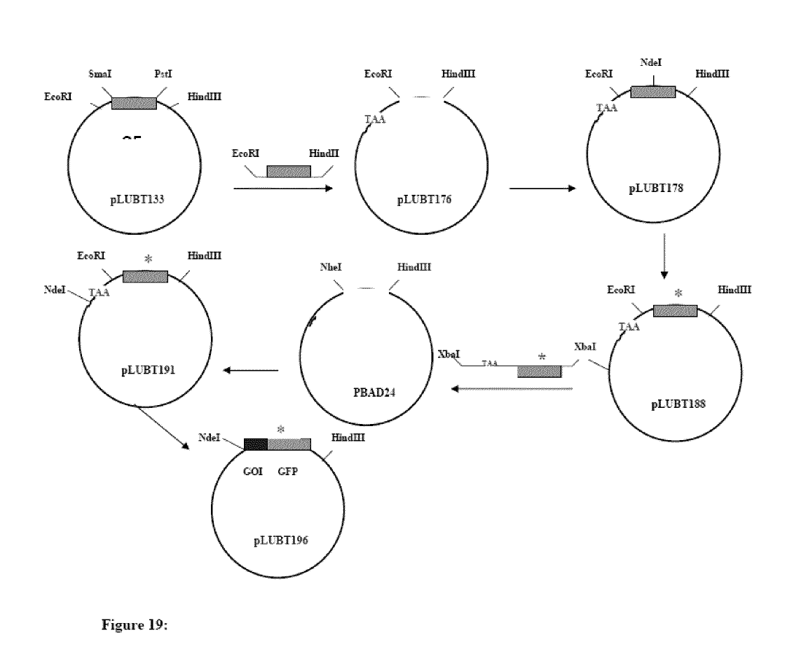

After overnight incubation at 30° C., plate was exposed under UV and some of the colonies were fluorescing upon UV exposure compared to others (FIG. 16). The glowing colonies are the clones of pLUBT191-Sak.

In order to confirm that the glowing colonies are recombinant clones and non-glowing as non-recombinants, samples of the glowing and non-glowing colonies were taken and subjected to PCR screening with GFP specific primers as well as Staphylokinase specific primers.

It was found that the glowing colonies give both the SAK specific and GFP PCR positive which are due to recombinant clones and non-recombinants, which do not glow under UV, show only GFP PCR but do not glow since they have TAA STOP codon at the N terminus of the GFP gene. Thus 5 glowing and 5 non-glowing colonies were selected and subjected to PCR with SAK specific and GFP specific primers.

The glowing cells are PCR positive with respect to SAK (FIG. 17) (Panel A, lanes 2-6) and non-glowing colonies are SAK PCR negative (FIG. 17) (Panel A, lanes 7-11). On the other hand all the colonies are positive for GFP PCR (FIG. 17) (Panel B).

Thus it was found that when the modified constructed vector pLUBT191 under arabinose promoter as a cloning vehicle was used, as a tool for foreign gene cloning so that only the recombinant clones would glow. Due to insertion of foreign genes in NdeI/EcoRI site, the STOP codon TAA gets removed and clones were selected directly based upon fluorescence or any other without the necessity of doing PCR, restriction analysis etc.

### Example 6

### Expression of the SAK-GFP Fusions in Non Amber Suppressing E. coli Strain BL21

The SAK and GFP glow positive clones #4 and #8 along with a non-glowing #5 clone DNA's were introduced in BL21(DE3) cells (a non amber suppressor E. coli B strain) and expression of SAK was induced with 13 mM L(+) arabinose. This result in expression of intact SAK gene alone in these cells, since SAK is cloned with amber STOP as GFP fusion. Expression of the heterologous proteins was analyzed on sodium dodecyl sulfate polyacrylamide gel electrophoresis (SDS-PAGE). It was found that the protein synthesis terminated at amber STOP and proteins of molecular size (15 kDa) was observed on SDSPAGE (FIG. 18).

The non-glowing clone did not express SAK since it is a non-recombinant with TAA at the N terminus of the GFP gene in pLUBT191

The applications of the present invention includes having following advantages as:
(1) The modified vector of the present invention provides consistent and reliable results for identification and screening of recombinant clones as they show colour or fluorescence after cloning whereas all other commercially available vectors show loss of colour or loss of fluorescence on cloning.
(2) In the present invention recombinants fluoresce under UV light or show color wherein non-recombinant does not fluoresce or color when introduced in the suppressor strain.
(3) False positives are completely avoided which is not possible with the commercially available vectors.
(4) Any other reporter gene found in the prior art could be used in place of GFP gene in the same vectors or different vectors.
(5) Different inducers can be used based upon the types of promoters used.
(6) The present invention also uses same vector for screening and expression in two different bacterial cell lines.
(7) The present invention can be used for cloning of any size genes since GFP is known to fluoresce when cloned as fusion protein with any size gene at the N terminus.
(8) The ligation mix using this vector need not be dephosphorylated as done in prior art since self ligated vector molecules without insert would never glow under UV light.
(9) This is a cost-effective process since it is not necessary to run control ligation (that is ligation with vector alone) at all, since only recombinants would glow and all our experiments show that this control is absolutely tight and not leaky.
(10) This vector would be cost-effective since it avoids the cost of restriction analysis, PCR etc which are routinely used for screening recombinants.
(11) This is a time saving method since only fluorescence or observation of color will differentiate between recombinant clones and non-recombinants.
(12) Further, Since GFP fluorescence is brightest when it is expressed in soluble form, the intensity of the fluorescence after cloning the foreign gene also indicates the extent of solubility of the fusion protein. This is the report of the first vector, which will indicate solubility of the foreign gene based on intensity of fluorescence.

### Example 6

### Qualitative Assessment of the Relative Solubility of the Proteins of Interest

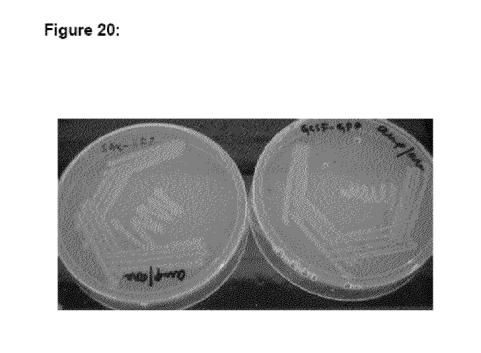

The solubility of the protein of interest would have an effect on the solubility of fusion protein and thereby affect the GFP fluorescence intensity. Thus from the relative fluorescence intensity of the fusion protein one can qualitatively assess the solubility propensity of the protein of interest. To validate this hypothesis, hGCSF was cloned (produced as insoluble aggregates in E. coli) and SAK (produced as soluble protein in E. coli) as GFP fusions with amber STOP. Both the constructs were introduced into competent LE392 E. coli cells (an amber suppressor strain) and plated on LB-agar semi-solid media containing 100 μg/ml ampicillin and 13 mM L(+) arabinose. Upon UV exposure a difference in fluorescence intensity depending on the solubility of the fusion protein was found. The results showed that (FIG. 20), SAK-fusion glow is brighter than hGCSF—fusion. This is due to better solubility of SAK protein over hGCSF.

1. A modified vector comprising a reporter gene having a STOP codon upstream of the multiple cloning site of the vector which is characterized in that the recombinant clones show fluoresce or show color in presence of inducer.

2. The modified vector as claimed in claim 1, wherein STOP codon is selected from TAA, TAG or TGA.

3. The modified vector as claimed in claim 1, wherein reporter genes is selected from the group comprising beta.-galactosidase gene, luciferase gene, alkaline phosphatase gene, secreted alkaline phosphatase gene, beta glucuronidase gene, all fluorescent protein gene (Green fluorescent protein, Yellow fluorescent protein, Red fluorescent protein, Enhanced green fluorescent protein, Orange fluorescent protein, Cyan fluorescent protein), substituted p-nitrophenyl phosphate and their variants.

4. The modified vector as claimed in claim 1, wherein vector is selected from plasmid, cosmid and phage.

5. A method for identification and selection of recombinant clones comprising the modified vector of claim 1, wherein the recombinant clones florescence or show color in a suitable suppressor strain of the STOP codon associated with the gene of interest.

6. The method for identification and expression of recombinant clone as claimed in claim 5, wherein STOP codon may be selected from TAA, TAG or TGA.

7. The method for identification and expression of recombinant clone as claimed in claim 5, wherein reporter genes may be, beta-galactosidase gene, luciferase gene, alkaline phosphatase gene, secreted alkaline phosphatase gene, beta glucuronidase gene, all fluorescent protein gene (Green fluorescent protein, Yellow fluorescent protein, Red fluorescent protein, Enhanced green fluorescent protein, Orange fluorescent protein, Cyan fluorescent protein), substituted p-nitrophenyl phosphate and their variants.

8. A method of preparation of a modified vector comprising a reporter gene having a STOP codon upstream of the multiple cloning site of a vector comprising:
(a) introduction of STOP codon upstream of multiple cloning site of the vector;
(b) amplification of reporter gene using primers;
(c) cloning of reporter gene in the vector;
wherein the modified vector when introduced in the non suppressor strain host does not fluoresce or show color upon induction.

9. The method of preparation of a modified vector as claimed in claim 8, wherein STOP codon is selected from TAA, TAG or TGA.

10. The method of preparation a modified vector as claimed in claim 8, wherein reporter genes may be, beta-galactosidase gene (beta-gal), luciferase gene (luc), alkaline phosphatase gene (AP), secreted alkaline phosphatase gene (SEAP), .beta.-glucuronidase gene (GUS), All fluorescent protein gene (Green fluorescent protein, Yellow fluorescent protein, Red fluorescent protein, Enhanced green fluorescent protein, Orange fluorescent protein, Cyan fluorescent protein), substituted p-nitrophenyl phosphate and their variants.

11. A method of preparation of recombinant clone comprising gene of interest and modified vector wherein the method comprises:
(a) Amplification of gene of interest using specific primers containing STOP codon different from STOP codon used with reporter gene
(b) Cloning the amplified gene of interest in the modified vector
(c) Transformation of cloned modified vector in the STOP codon suppressor host cell wherein the STOP codon suppressor host cell is specific for STOP codon used with the gene of interest wherein the recombinant clones either fluorescence or show color depending upon the reporter gene used.

12. The method of preparation a recombinant clone as claimed in claim 11, wherein STOP codon is selected from TAA, TAG or TGA different from STOP codon used with reporter gene.

13. The method of preparation a recombinant clone as claimed in claim 11, wherein reporter genes is selected from beta.-galactosidase gene (.beta.-gal), luciferase gene (luc), alkaline phosphatase gene (AP), secreted alkaline phosphatase gene (SEAP), .beta.-glucuronidase gene (GUS), All fluorescent protein gene (Green fluorescent protein, Yellow fluorescent protein, Red fluorescent protein, Enhanced green fluorescent protein, Orange fluorescent protein, Cyan fluorescent protein), substituted p-nitrophenyl phosphate and their variants.

14. The method of preparation of recombinant clone as in claim 11, wherein the recombinant clones when expressed in non suppressor host cell do not fluoresce or show color & yields protein of interest.

15. A kit for identification and expression of recombinant clones comprising modified vector wherein the modified vector comprises of reporter gene carrying STOP codon.

16. A kit for identification and expression of recombinant clones wherein as in claim 15, STOP codon is selected from TAA, TAG or TGA.

17. A kit for identification and expression of recombinant clones wherein as in claim 15, reporter genes may be, beta.-galactosidase gene luciferase gene, alkaline phosphatase gene, secreted alkaline phosphatase gene, beta.-glucuronidase gene, all fluorescent protein gene (Green fluorescent protein, Yellow fluorescent protein, Red fluorescent protein, Enhanced green fluorescent protein, Orange fluorescent protein, Cyan fluorescent protein), substituted p-nitrophenyl phosphate and their variants.

18. A kit for identification and expression of recombinant clones wherein as in claim 15, further comprise of gene of interest carrying a STOP codon different from STOP codon used with reporter gene.

19. A kit for indicating the solubility of foreign protein expressed as a fusion protein using the fluorescing or colored recombinant clone wherein the intensity of the color is dictated by the solubility index of the foreign protein that is expressed using a recombinant clone identified and screened using modified vector of claim 1.
